(19) United States
(12) Patent Application Publication
(10) Pub. No.: US 2016/0073956 A1
(43) Pub. Date: Mar. 17, 2016

(54) Secure tracking apparatus strap

(71) Applicant: Ohanes D. Ghazarian, Henderson, NV (US)

(72) Inventor: Ohanes D. Ghazarian, Henderson, NV (US)

(21) Appl. No.: 14/121,530

(22) Filed: Sep. 15, 2014

(51) Int. Cl. A61B 5/00; A61B 5/0205; H04Q 9/00

(52) U.S. Cl. CPC A61B 5/6843; H04Q 9/00; A61B 5/02055; A61B 5/0059; A61B 5/4875; A61B 5/6831; A61B 5/01

(57) ABSTRACT

A tracking apparatus having a strap containing sensor(s) operatively connected at least two sides of a tamper detection tracking apparatus, and at least one or more of the sensors are configured to communicate with the apparatus processor through the strap in order to complete the tamper detection circuitry. The tamper detection strap sensors configured to detect body presence within the proximity of the strap, for the apparatus processor to determine the apparatus is securely attached to a person be monitored. The tracking apparatus contains a positioning locator receiver and a wireless communication module used to communicate with a monitoring station. Wherein when the tamper detection strap is cut, disconnected or removed the processor stop receiving signals from one or more sensors, the tracking apparatus transmits at list one identifiable tamper detection signal, location information or both the tamper detection and location information signal to a monitoring station.

This application benefits prior U.S. provisional application 61/960,814

In the past many different methods have been used to securely attach a tracking device to a person or an object to be monitored, one such commonly known system is use of a conductive or optical tamper detection straps, used for securely attach a tamper detection tracking apparatus to a person limp or an object. If and when the person to be monitored, tamper's with the tracking apparatus housing, cuts or remove the strap, the tracking device transmits a tamper detection signal to a monitoring station.

Even though prior art teachings are useful in the art, however the prior art teachings conductivity or optical strap cannot define if the tracking device is securely attached to a person or an object, there for a monitoring station personnel cannot verify a subject matter to be monitored is a living person or an object. There for one can attached the tracking apparatus to a person or an object, and monitoring station personnel will never be aware if the subject matter to be tracked is a person or an object, and may falsely track and object thinking he or she is tracking a person. Such prior art teaching found in U.S. Pat. No. 7,034,683, and RE 44,275 to Ghazarian. U.S. Pat. No. 6,014,080 to Layson. And U.S. Pat. No. 5,867,103 to Taylor. Wherein the conductive or optical tamper detection strap is used to secure the tracking apparatus to the person or an object, where the monitoring station operator cannot define subject matter being monitored is a person or an object.

It is accordingly the primary objective of the present invention, wherein the tracking apparatus having a positioning locator receiver used for receiving location coordinate information, a radio frequency module used for establishing radio frequency communication with a monitoring station, and a tamper detection strap containing sensor(s) used for securely attach the tamper detection tracking apparatus to a persons or an object. The present invention tamper detection strap contain sensors configured to detect body presence within the proximity of the tamper detection strap, and accordingly the tracking apparatus processor determines the tracking apparatus is securely attached to a person or an object to be monitored. And the present invention tracking apparatus housing further may contain sensor(s) configured to detect body presence, and accordingly the tracking apparatus processor determines the tracking apparatus is securely attached to a person or to an object to be monitored.

It is accordingly the objective of the present invention to utilize a tracking apparatus having a tamper detection strap containing at list one or more of sensor(s) or certain combination of sensors, and the tracking apparatus housing utilizes at least one or more following sensor(s) configured to detect human body or an object presence;

Body resistance measuring sensor,

Body temperature measuring sensor,

Body conductivity reader sensor,

Body surface optical reflection sensor,

Body blood glucoses measuring sensor,

Heart pulse detection sensor,

Body moister detection sensor,

The tracking apparatus tamper detection strap sensor(s) are operatively connected at least two sides of the tamper detection tracking apparatus housing, and at least one or more sensors are configured to communicate through the strap in order to complete the tamper detection circuitry, wherein when the tracking apparatus is detached, or the tamper detection strap is cut, disconnected or removed from subject to be monitored, the processor stop receiving signals from one or more sensors or combination of sensors, the tracking apparatus then transmits at list one identifiable tamper detection signal through the radio frequency module to a monitoring station, and send positioning location information of the tracking apparatus to a monitoring station.

### SUMMERY OF THE INVENTION

The present invention, utilizes a tracking apparatus having a positioning locator receiver used for receiving location coordinate information, a radio frequency module used for establishing radio frequency communication with a monitoring station, and a tamper detection strap containing sensor(s) used for securely attach the tamper detection tracking apparatus to a persons or an object. The present invention tamper detection strap contain sensors configured to detect human body or object presence, and accordingly the tracking apparatus processor determines the tracking apparatus is securely attached to a person or an object to be monitored. And the present invention tracking apparatus housing further may contain a sensor(s) configured to detect human body or object presence, and accordingly the tracking apparatus processor determines the tracking apparatus is securely attached to a person or an object to be monitored.

The tracking apparatus body detection sensors may be of the following;

Body resistance measuring sensor,

Body temperature measuring sensor,

Body conductivity reader sensor,

Body surface optical reflection sensor,

Body blood glucoses measuring sensor,

Heart pulse detection sensor,

Body moister detection sensor,

Wherein when the tracking apparatus is detached, or the tamper detection strap is cut, disconnected or removed from subject to be monitored, the processor stop receiving signals from one or more sensors or combination of sensors, the tracking apparatus then transmits at list one identifiable tamper detection signal through the radio frequency module to a monitoring station, and send positioning location information of the tracking apparatus to a monitoring station.

### BRIEF DESCRIPTION OF THE DRAWINGS

FIG. 1. Is the block Diagram.

FIG. 2. Is a drawing of a tracking apparatus wherein the tracking apparatus is attached to a person or an object to be monitored by a tamper detection strap having body presence detection sensors.

FIG. 3. Is the drawing of a tracking apparatus having a body presence detection sensor attached to a subject to be monitored with a Velcro or adhesive tape.

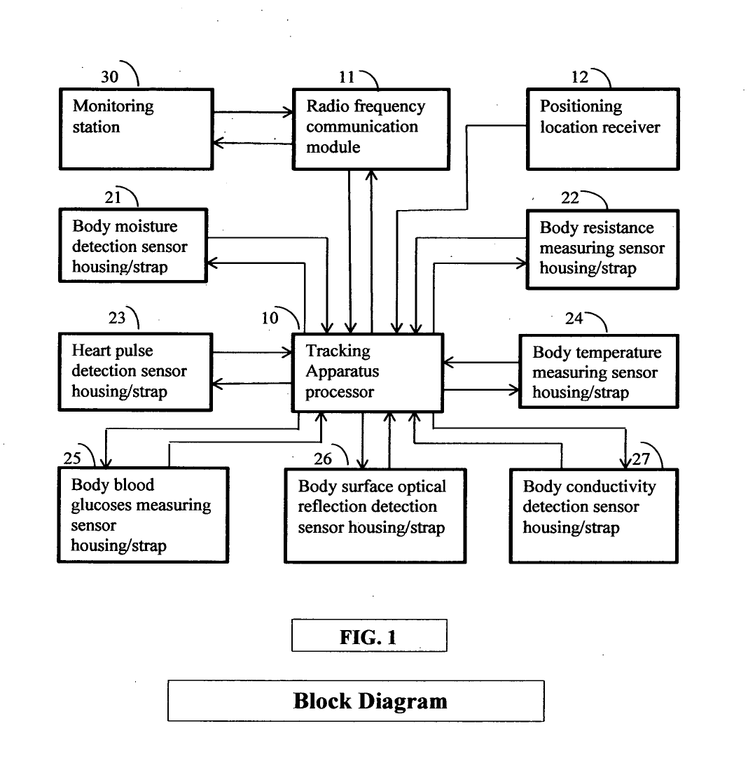

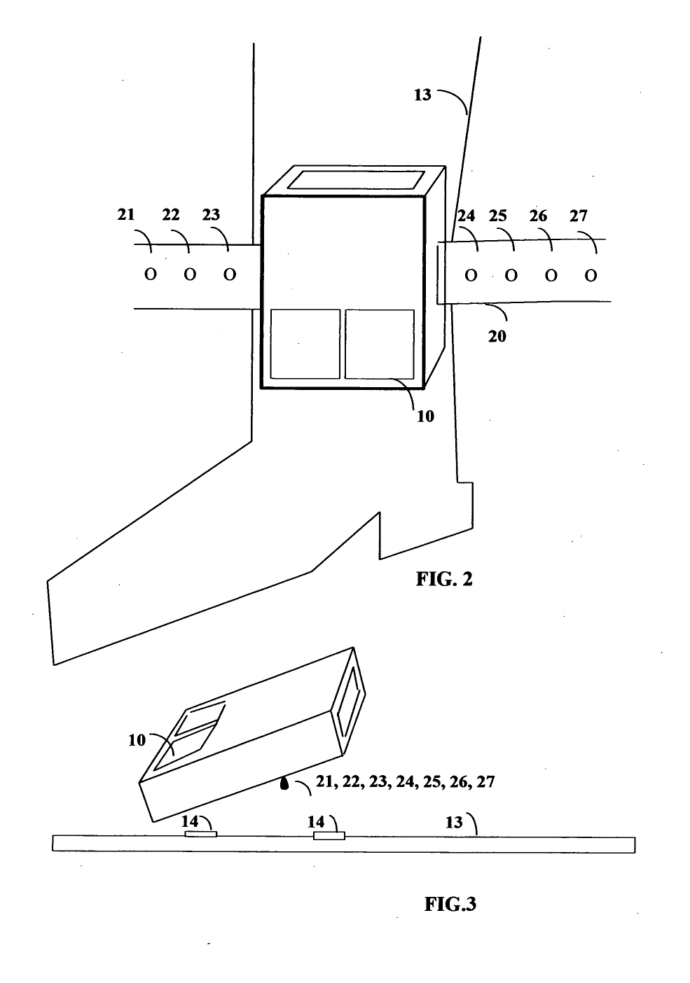

### DETAILED DESCRIPTION OF THE INVENTION

Wherein FIGS. 1 and 2, the tracking apparatus 10 of the present invention having a positioning locator receiver 12, used for receiving location coordinate information of the tracking apparatus 10, a radio frequency communication module 11, used for establishing radio frequency communication between the tracking apparatus 10 and a monitoring station 30, or other communication devices, and a tamper detection strap 20 containing sensor(s) 21, 22, 23, 24, 25, 26, and 27 used for securely attach the tamper detection tracking apparatus 10 to a persons or an object. The present invention tamper detection strap 20 contain sensors 21, 22, 23, 24, 25, 26, and 27 configured to detect body presence within the close proximity of the tamper detection strap 20, and accordingly the tracking apparatus processor 10 determines the tracking apparatus is securely attached to a person or an object to be monitored. And the present invention tracking apparatus FIG. 3 housing 10 further may contain sensor(s) 21, 22, 23, 24, 25, 26, and 27, configured to detect human body presence within close proximity 13 of a person or object attached too, and accordingly the tracking apparatus processor 10 determines the tracking apparatus is securely attached with the use of Velcro tape or adhesive 14 to a person or an object 13 to be monitored.

The present invention utilizes a tracking apparatus 10 having a tamper detection strap 20 containing at list one or more of sensor(s) or may utilize certain combination of sensors, 21, 22, 23, 24, 25, 26, and 27, and the tracking apparatus housing 10 may utilizes at least one or more following sensor(s) 21, 22, 23, 24, 25, 26, and 27 configured to detect human body or an object presence within close proximity of the sensors wherein it is attached to; the sensors may comprises of;

Body moisture detection sensor 21,

Body resistance measuring sensor 22,

Heart pulse detection sensor 23,

Body temperature measuring sensor 24,

Body blood glucoses measuring sensor 25,

Body surface optical reflection sensor 26,

Body conductivity reader sensor 27,

The tracking apparatus 10 tamper detection strap sensor(s) 21, 22, 23, 24, 25, 26, and 27 are operatively connected at least two sides of the tamper detection tracking apparatus housing 10, and at least one or more sensors are configured to communicate through the strap 20 in order to complete the tamper detection circuitry. Wherein when the tracking apparatus 10 is detached, or the tamper detection strap 20 is cut, disconnected or removed from subject to be monitored 13, the processor 10 stop receiving signals from one or more sensors or combination of sensors, 21, 22, 23, 24, 25, 26, and 27, the tracking apparatus processor 10 then transmits at list one identifiable tamper detection signal through the radio frequency module 12 to a monitoring station 30, and send positioning location information of the tracking apparatus to a monitoring station 30.

1. A tracking apparatus attached to a person or an object to be monitored, said apparatus containing;
a battery or other power means to supply power to said tracking apparatus,
a positioning locator receiver, used to receive location information of said tracking apparatus, a wireless communication module, used to establish communication with a monitoring station, or establish communication with other communication devices,
a processor, and
a tamper detection strap containing at least one or more, or any combination of sensor(s) used for securely attaching said tamper detection tracking apparatus to a person(s) or object(s) to be monitored, said sensor(s) is used to detect human body or object presence near proximity of said apparatus and feed said detected signal(s) to said apparatus processor, to determines said apparatus is securely attached to a person or an object to be monitored, said sensors comprising;
a),
d) Body or object detection optical sensor,
e) Body blood glucose measuring sensor,
f) Heart pulse detection sensor,
said tamper detection strap sensor(s) are operatively connected at least two sides of the tamper detection tracking apparatus housing, and at least one or more sensors are configured to communicate through the strap with said apparatus processor in order to complete the tamper detection circuitry, and said apparatus processor based on said received signals from said sensor(s) determines the tracking apparatus is attached to a person or to an object to be monitored,
wherein when the said tracking apparatus strap is tampered, cut or detached from said person or object to be monitored, said processor stop receiving signals or receive alternate signals from said one or more sensors, the tracking apparatus processor in response transmits at least one signal comprising an identification of the apparatus, the identification of the apparatus and a condition of the apparatus, the identification of the apparatus and positioning location information of the apparatus, or the identification of the apparatus, the condition of the apparatus, and the global positioning location information of the apparatus to said monitoring station or to other communication devices, to allow the monitoring station or other communication devices, based on at least one received signal, to determine the condition of the apparatus, to inquire the apparatus to send at least one or more global positioning location information of the apparatus.

2. A tracking apparatus attached to a person or object to be monitored, said apparatus containing;
a battery or other power means to supply power to the tracking apparatus,
a positioning locator receiver, used to receive location information of said tracking apparatus,
a wireless communication module, used to establish communication with a monitoring station, or establish communication with other communication devices,
a processor, and
at least one or more, or any combination of detection sensor(s) used for securely attaching said tamper detection tracking apparatus to a person(s) or object(s) to be monitored, said sensor(s) is used to detect human body or object presence near proximity of said apparatus and feed said detected signal(s) to said apparatus processor, to determines said apparatus is securely attached to a person or an object to be monitored, said sensors comprising;
a),
d) Body or object detection optical sensor,
e) Body blood glucoses-measuring sensor,
f) Heart pulse detection sensor,
g),
said tracking apparatus is securely attached to a persons or an object to be monitored, said sensor(s) operatively is connected to said tracking apparatus processor, in order to complete the tamper detection circuitry, and said apparatus processor based on said received signals from said sensor(s) determines said tracking apparatus is attached to a person or to an object to be monitored,
wherein when the said tracking apparatus is detached from said person or object to be monitored, said processor stop receiving signals or receive alternate signal(s) from said one or more sensor(s), said tracking apparatus processor in response transmits at least one signal comprising an identification of the apparatus, the identification of the apparatus and a condition of the apparatus, the identification of the apparatus and positioning location information of the apparatus, or the identification of the apparatus, the condition of the apparatus, and the global positioning location information of the apparatus to said monitoring station or to other communication devices, to allow the monitoring station or other communication devices, based on at least one received signal, to determine the condition of the apparatus, to inquire the apparatus to send at least one or more global positioning location information of the apparatus.

3. A tracking apparatus securely attached to a person or an object to be monitored, said
apparatus containing;
a battery or other power means to supply power to said tracking apparatus,
a positioning locator receiver, used to receive location information of said tracking apparatus,
a wireless communication module, used to establish communication with a monitoring station, or establish communication with other communication devices,
a processor, and
a tamper detection strap containing at least one combination or more sensor(s) used for securely attaching said tamper detection tracking apparatus to a person(s) or object(s) to be monitored, said sensors is used to detect human body or object presence near proximity of said apparatus and feed said detected signals to said apparatus processor, for said to ascertain and to distinct said apparatus is securely attached to a person or attached to an object to be monitored, said sensors comprising;
a) Body or object resistance measuring sensor,
b) Body or object temperature measuring sensor,
c) Body or object conductivity measuring sensor,
d) Body or object detection optical sensor,
e) Body blood glucose measuring sensor,
f) Pulse detection sensor,
g) Body moister detection sensor,
said tamper detection strap sensors are operatively connected at least two sides of the tamper detection tracking apparatus housing, and at least combination or more sensors are configured to communicate through the strap with said apparatus processor in order to complete the tamper detection circuitry, and said apparatus processor based on said received signals from said sensors determines the tracking apparatus is attached to a person or to an object to be monitored,
wherein when the said tracking apparatus strap is tampered, cut or detached from said person or object to be monitored, said processor stop receiving signals or receive alternate signals from said sensors, the tracking apparatus processor in response transmits at least one signal comprising an identification of the apparatus, the identification of the apparatus and a condition of the apparatus, the identification of the apparatus and positioning location information of the apparatus, or the identification of the apparatus, the condition of the apparatus, and the global positioning location information of the apparatus to said monitoring station or to other communication devices, to allow the monitoring station or other communication devices, based on at least one received signal, to determine the condition of the apparatus, to inquire the apparatus to send at least one or more global positioning location information of the apparatus.

4. A tracking apparatus securely attached to a person or object to be monitored, said apparatus containing;
a battery or other power means to supply power to the tracking apparatus,
a positioning locator receiver, used to receive location information of said tracking apparatus,
a wireless communication module, used to establish communication with a monitoring station, or establish communication with other communication devices,
a processor, and
at least one combination or more detection sensor(s) used for securely attaching said tamper detection tracking apparatus to a person(s) or object(s) to be monitored, said sensors is used to detect human body or object presence near proximity of said apparatus and feed said detected signals to said apparatus processor, for said processor to ascertain and to distinct said apparatus is securely attached to a person or attached to an object to be monitored, said sensors comprising; p1 a) Body or object resistance measuring sensor,
b) Body or object temperature measuring sensor,
c) Body or object conductivity measuring sensor,
d) Body or object detection optical sensor,
e) Body blood glucose measuring sensor,
f) Pulse detection sensor,
g) Body skin moister detection sensor,
said tracking apparatus is securely attached to a persons or an object to be monitored, said sensor(s) operatively is connected to said tracking apparatus processor, in order to complete the tamper detection circuitry, and said apparatus processor based on said received signals from said sensor(s) determines said tracking apparatus is attached to a person or to an object to be monitored,
wherein when the said tracking apparatus is detached from said person or object to be monitored, said processor stop receiving signals or receive alternate signal(s) from said one or more sensor(s), said tracking apparatus processor in response transmits at least one signal comprising an identification of the apparatus, the identification of the apparatus and a condition of the apparatus, the identification of the apparatus and positioning location information of the apparatus, or the identification of the apparatus, the condition of the apparatus, and the global positioning location information of the apparatus to said monitoring station or to other communication devices, to allow the monitoring station or other communication devices, based on at least one received signal, to determine the condition of the apparatus, to inquire the apparatus to send at least one or more global positioning location information of the apparatus.
